(19) United States
(12) Patent Application Publication
(10) Pub. No.: US 2018/0159193 A1
(43) Pub. Date: Jun. 7, 2018

(54) E-PLANE WAVEGUIDE CIRCULATOR FOR OPERATION ABOVE MAGNETIC RESONANCE

(71) Applicant: Apollo Microwaves Ltd., Dorval (CA)

(72) Inventors: Louis-Philippe Carignan, Pincourt (CA); Vasilios (Bill) Tsounis, Pincourt (CA)

(73) Assignee: Apollo Microwaves Ltd., Dorval, QC (CA)

(21) Appl. No.: 15/576,391

(22) PCT Filed: May 27, 2015
(86) PCT No.: PCT/CA2015/050481

§ 371 Date: Nov. 22, 2017

(51) Int. Cl. H01P 1/383; H01P 7/10; H01F 7/20; H05K 7/20; A61N 5/10

(52) U.S. Cl. CPC H01P 1/383; H01P 7/10; H01F 7/202; A61N 2005/1089; A61N 5/10; A61N 2005/1087; H05K 7/2039

(57) ABSTRACT

An E-plane waveguide circulator for use in a high power microwave circuit is provided. The circulator has three or more waveguide ports intersecting at a junction, wherein the junction has an upper inner surface and a lower inner surface positioned in an opposing relationship to said upper inner surface. The circulator further includes at least one radial composite resonator positioned within the junction, the resonator being comprised of a composite made of a centrally disposed ferrite element and a solid dielectric layer disposed concentrically with and adjacent externally to said centrally disposed ferrite element. In use, a magnetic field source applies an external magnetic field to the radial composite resonator, the external magnetic field having a magnitude above the magnetic resonance associated with the radial composite resonator.

### FIELD OF THE INVENTION

The present invention relates to the field of passive microwave components and, more specifically, to a space efficient configuration for an E-plane waveguide circulator configured for operation above magnetic resonance and having one or more ferrite resonators.

### BACKGROUND

Waveguide circulators are known in the art for handling RF waves. Typically, waveguide circulators include three ports (although more or less ports are possible) and are used for transferring wave energy in a non-reciprocal manner, such that when wave energy is fed into one port, it is transferred in one direction to a next port only. A common use for waveguide circulators is to transmit energy from a transmitter to an antenna during transmitting operations, and to transmit energy from an antenna to a receiver during receiving operations.

In order to enable the non-reciprocal energy transfer, the waveguide circulators include ferrite resonators to which are applied a magnetic field via one or more permanent magnets or electromagnets. E-plane and H-plane waveguide circulators are two configurations of such waveguide circulators. H-plane waveguide circulators are commonly used in equipment for ground and space applications and radar applications. While E-plane circulators are known and have been the object of technical publications, their use is not as widespread as H-plane waveguide circulators in practical applications.

An example of a typical H-plane waveguide circulator 300 is illustrated in FIGS. 1A and 1B. In this example, the waveguide circulator 300 has three waveguide ports 302, 304 and 306 that meet at a common junction 308. Shown in FIG. 1B is a side view of the waveguide circulator 300 with a view into waveguide port 306. Positioned within the junction 308 of the waveguide circulator 300 is a pair of gyromagnetic members 305 and 307, which are also referred to as “ferrite elements” or “resonators” and are typically made of a ferrite material. The ferrite elements 305 and 307 are positioned within the junction 308, generally upon mounting pedestals 350 and 370 located on opposing inner surfaces 303 and 303′ of the junction 308, such that they are centrally disposed and arranged generally symmetrically, with respect to the three waveguide ports 302, 304 and 306.

During operation, the ferrite elements 305 and 307 are subjected to the influence of a magnetic field that is generated by one or more magnets or electromagnets (not shown), which can be positioned on outside surfaces of the junction 308 above and below the ferrite elements 305 and 307. The magnetic field that is generated is a unidirectional magnetic field, represented by arrow 309 in FIGS. 1A and 1B, such that wave energy entering each waveguide ports 302, 304 and 306 will move in a counter-clockwise direction (or clockwise depending on the direction of the magnetic field) towards its neighboring waveguide port. As such, the waveguide circulator 300 is a non-reciprocal transmitter of electromagnetic wave energy propagating in the waveguides.

In certain applications it may be desirable to have a waveguide circuit including waveguide circulators that can handle high peak power level RF waves.

However, known H-plane waveguide circulators tend to operate below typically 1 to 3 MW peak-power RF waves, which may restrict their use in certain applications requiring high peak power handling capabilities. Factors limiting the power handling capability of the circulator may include, without being limited, to the size of the gap between the ferrites, the temperature of the ferrites, the pressure and the material between the ferrites (usually pressurized sulfur hexafluoride or pressurized air). In particular, for H-plane circulators, the peak power level is limited to a large extent because the gap between the ferrite elements in such circulators is relatively small. When the gap becomes too small, the electric field becomes large for the limited space between the ferrite elements and there can be a corona discharge in that space. Such discharge can cause damage not only to the circulator but, in some cases, also to the high power source that generated the signals propagating through the circulators. Therefore, and as can be appreciated, there is a limit to the peak power handling of H-plane circulators, dictated by the size of the gap between the ferrite elements as well as other factors.

Another deficiency with conventional H-plane waveguide circulators is that those devices suitable for handling high peak power level RF waves tend to be bulky and tend require a large amount of real-estate space when installed. This renders them impractical for certain applications.

In light of the above, there is a need in the industry for an improved waveguide circulator that alleviates, at least in part, the deficiencies with existing waveguide circulators.

### SUMMARY

In accordance with a first aspect, an E-plane waveguide circulator for use in a high power microwave circuit is provided. The waveguide circulator comprises at least three waveguide ports intersecting at a junction, wherein the junction has an upper inner surface and a lower inner surface positioned in an opposing relationship to the upper inner surface. The waveguide circulator also comprises a radial composite resonator positioned within said junction, the resonator being comprised of a composite made of a centrally disposed ferrite element and a solid dielectric layer disposed concentrically with and adjacent externally to the centrally disposed ferrite element. In use, an external magnetic field source is used to apply an external magnetic field to the radial composite resonator, the external magnetic field having a magnitude above a magnetic resonance associated with the radial composite resonator.

The E-plane circulator proposed in the present document is operated using an external magnetic field of a magnitude above magnetic resonance. Below magnetic resonance, there is a subsidiary resonance that absorbs RF energy, by the excitation of spin waves, which limits the maximum operating power level of the circulator. By operating above resonance, the impact on energy absorption by the ferrite due to an overlap between a subsidiary resonance and a main resonance associated with the resonators can be avoided. As a result, by using an E-plane circulator of the type suggested in the present application and operating it above resonance, a circulator having a higher peak power handling capability can be obtained.

In addition, the use of one of more radial composite resonators having a dielectric layer disposed concentrically with and adjacent externally to a centrally disposed ferrite element as described above may allow for improved dissipation of energy absorbed by the ferrite to the body of the circulator. Such improved dissipation helps increase the average wave power level handling capability of the resonators and thus increases the average wave power level handling capability of the circulator as a whole.

The specific shape of the ferrite element and solid dielectric layer may vary from one implementation to the other. For example, in some specific implementations, the ferrite element may be a ferrite disk and the solid dielectric layer may be a dielectric ring shaped to surround the ferrite disk. In some other specific implementations, the ferrite element may have a triangular shape and the solid dielectric layer may have a complementary triangular inner surface for surrounding the periphery of the ferrite element.

Optionally, the radial composite resonator may further include a dielectric stack covering at least in part a top surface of the ferrite element. This dielectric stack may assist in dissipating heat from the resonator, thus further increasing the average power handling capability of the circulator.

In specific implementations, the resonator may be positioned on one of the upper inner surface and the lower inner surface of the junction. In some specific implementations, the radial composite resonator may be a first radial composite resonator and the circulator may comprise a second radial composite resonator positioned on the other one of the upper inner surface and the lower inner surface of the junction in a spaced-apart opposing relationship with the first radial composite resonator. The second radial composite resonator may be similarly configured to the first radial composite resonator and may be comprised of a composite made of a centrally disposed ferrite element and a solid dielectric layer disposed concentrically with and adjacent externally to the centrally disposed ferrite element.

In specific implementations, the resonator may be positioned on a mounting pedestal formed on one of the upper inner surface and the lower inner surface of the junction. In some specific implementations, the radial composite resonator may be a first radial composite resonator and the mounting pedestal may be a first mounting pedestal, and the circulator may comprise a second radial composite resonator positioned on a second mounting pedestal formed on the other one of the upper inner surface and the lower inner surface of the junction in a spaced-apart opposing relationship with the first radial composite resonator. Optionally, the resonator may be positioned within a recess formed within the mounting pedestal(s), the resonator including a first portion projecting into the junction and a second portion extending into the recess.

In some implementations, a cooling module may be provided including circulation piping for circulating a coolant near the junction to assist in dissipating heat from the resonator, thus further increasing the average power handling capability of the circulator. In a non-limiting example in which a pulse-type RF field is applied to the circulator, heat is generated and is to be dissipated between the pulses. Typically in such cases the RF field would be applied during a short period of time, typically a couple of microseconds, and then turned off for a few milliseconds. For example, an 8 MW pulse of 5 microseconds on, and 5 milliseconds off will give an average power of about 8 kW. It is the average power level that will heat up the ferrites, and therefore needs to be dissipated to prevent the ferrites from overheating. By using a coolant, the rate at which the heat is dissipated can be increased thus increasing the average power handling capability of the circulator.

Other aspects and features of the present invention will become apparent to those ordinarily skilled in the art upon review of the following description of specific embodiments of the invention in conjunction with the accompanying drawings.

### BRIEF DESCRIPTION OF THE DRAWINGS

A detailed description of specific embodiments of the present invention is provided herein below with reference to the accompanying drawings in which:

FIG. 1 A is a perspective view of an H-plane waveguide circulator in accordance with a typical configuration.

FIG. 1B is a side plan view of the H-plane waveguide circulator of FIG. 1A, exposing the interior of the waveguide circulator.

FIG. 2 is a perspective view of an E-plane waveguide circulator in accordance with a non-limiting example of implementation of the present invention;

FIG. 3 is a top plan view of the waveguide circulator of FIG. 2;

FIG. 4 is a front elevation view of the waveguide circulator of FIG. 2 showing one of the waveguide ports of the waveguide circulator;

FIG. 5 is a cross-sectional view of the waveguide circulator of FIG. 2 taken along line 4-4 in FIG. 3;

FIG. 6 is a perspective view of a bottom portion of the waveguide circulator of FIG. 2 showing waveguide ports of the waveguide circulator meeting at a junction;

FIG. 7 is a top view of the bottom portion of the waveguide circulator shown in FIG. 6;

FIGS. 8 and 9 are respectively a top plan view and a side view of a radial composite resonator for use in the waveguide circulator of FIG. 2 in accordance with a specific implementation;

FIG. 10A is a perspective view of a waveguide circulator in accordance with an alternative embodiment;

FIGS. 10B, 10C, 10D and 10E respectively are a front view, a top view, a side view and a rear view of the waveguide circulator of FIG. 10A;

FIGS. 11 and 12 respectively are a perspective view and a top view of a bottom portion of the waveguide circulator of FIG. 10A showing waveguide ports of the waveguide circulator meeting at a junction;

FIG. 13 is a graph showing energy loss as a function of a magnetic field magnitude applied to a waveguide circulator of the type shown in either FIG. 2 or FIG. 10A;

FIG. 14 is a conceptual schematic representation of magnetic dipole moments inside a ferrite element aligned in the direction of an external magnetic DC field produced by permanent magnets and/or an electromagnet.

FIGS. 15A and 15 B are conceptual schematic representations of magnetic fields produced by magnetic dipole moments in magnetic materials. FIG. 15A shows a field produced by a disk-shaped structure and FIG. 15B shows a field produced by a cylinder-shaped structure;

FIG. 16 is a conceptual schematic illustrating the precession of a magnetic moment of a ferrite element subjected to a DC field produced by magnets and an RF magnetic field produced by microwaves travelling in a waveguide;

FIGS. 17A and 17B are graphs showing real and imaginary parts of RF permeability associated to a ferrite element. FIG. 17A shows the RF permeability as a function of magnitude of a DC magnetic field and FIG. 17B shows the RF permeability as a function a frequency associated with a microwave signal travelling in a waveguide in which the ferrite element is located.

FIGS. 17C is a graph showing real and imaginary parts of the effective RF permeability μ_{eff} permeability associated to a ferrite element as a function a frequency associated with a microwave signal travelling in a waveguide in which the ferrite element is located.

FIG. 18 is a graph showing energy loss relative to magnitudes of a magnetic field applied to a ferrite element showing subsidiary resonance effects at different power levels.

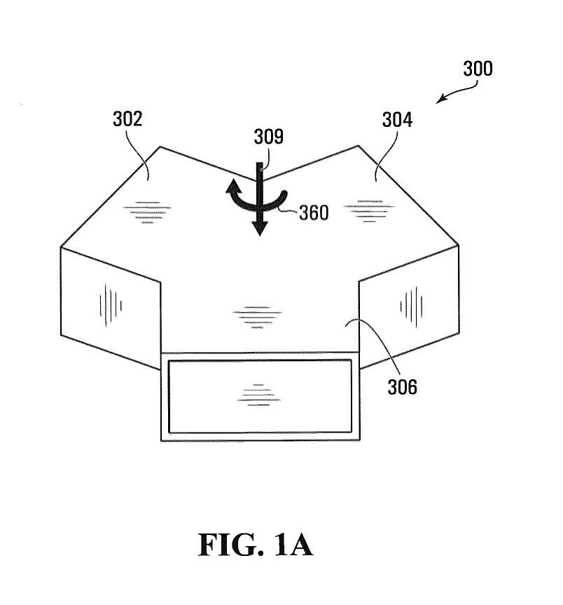

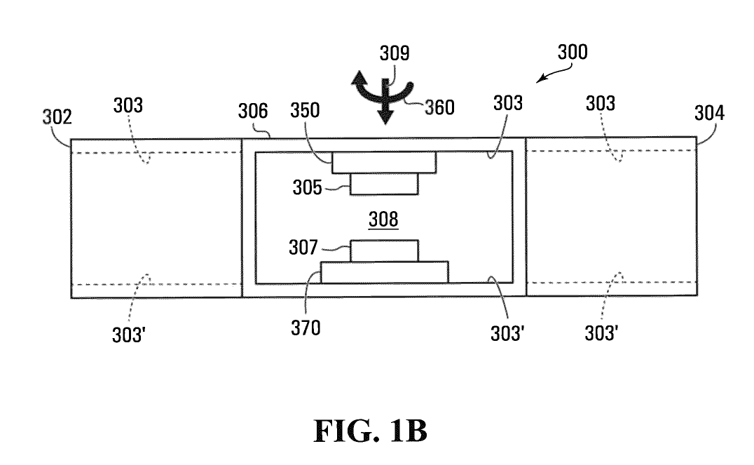

In the drawings, embodiments of the invention are illustrated by way of example. It is to be expressly understood that the description and drawings are only for the purpose of illustrating certain embodiments of the invention and are an aid for understanding. They are not intended to be a definition of the limits of the invention.

### DETAILED DESCRIPTION

For the purpose of clarity in the present description, it is to be understood that the meaning of above or below resonance is intended to refer to above or below magnetic resonance with respect to the magnetic field, not the operating frequency.

Specific examples of waveguide circulators will now be described to illustrate the manner in which the principles of the invention may be put into practice. Such waveguide circulators may have particular utility in satellite communications equipment encompassing both ground and space segments, as well as in the radar and medical fields.

Typical 3-port circulators are either H-plane or E-plane. While average power handling capability may be higher for H-plane circulator when compared to E-plane circulator, E-plane circulators can generally handle higher peak powers. The high peak power handling capability is provided in part by the presence of a larger gap between the resonators in an E-plane circulator when compared to the gap in an H-plane circulator. In addition, E-plane circulators tend to be more compact in comparison to their counterpart conventional H-plane circulators due to their geometry. Table 1 summarizes some differences between the use of E or H-plane configurations.

TABLE 1
Some distinctions between E and H-plane junction circulators

| Property | H-Plane | E-Plane |
|---|---|---|
| Bandwidth | Very Large | Small to average |
| Return loss | Very good | Very good |
| Peak power handling | Good | Very good |
| Average power handling | Very good | Good |
| Compactness | Good | Very good |

The present document presents an improved design for an E-plane circulator, rather than H-plane circulator, in order to provide a generally compact circulator that has high peak power handling capabilities. The more compact design may make such proposed E-plane circulator more practical for certain applications in which it is desirable to reduce the amount of space used.

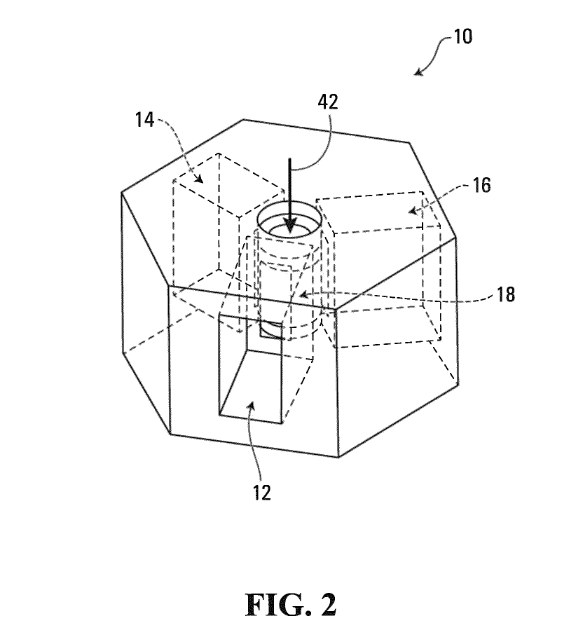

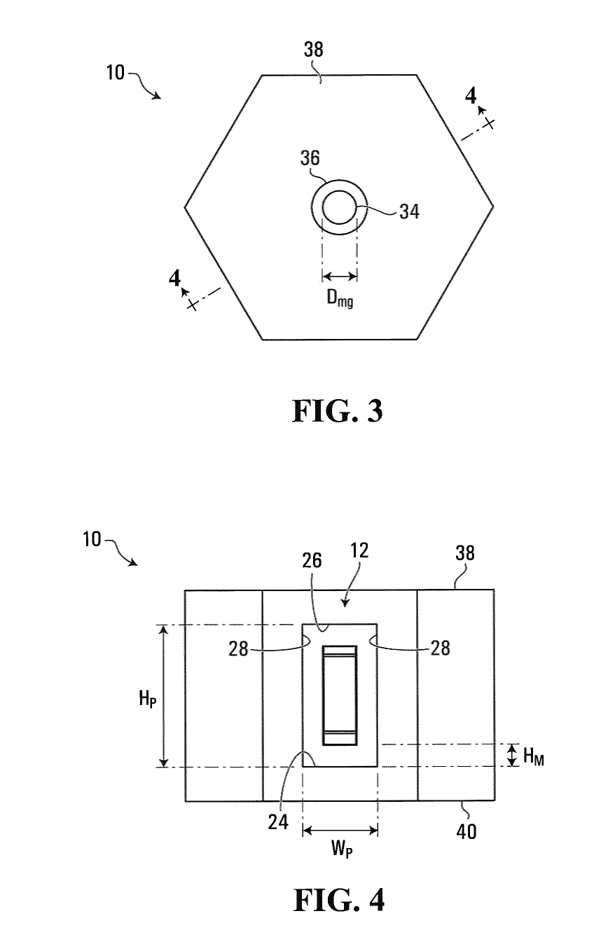

Shown in FIGS. 2 through 5 is an E-plane waveguide circulator 10 in accordance with a non-limiting example of implementation of the present invention. As shown, the waveguide circulator 10 comprises three waveguide ports 12, 14 and 16 that meet at a common junction 18. In the embodiment shown, the three waveguide ports 12, 14 and 16 are evenly spaced at 120° angles in relation to each other. Although three evenly spaced waveguide ports 12, 14 and 16 are shown in these FIGS. 2, alternative implementations of waveguide circulators may include more or less than three waveguide ports, as well as waveguide ports that are not evenly spaced.

Positioned within the junction 18 of the waveguide circulator 10 is a pair of radial composite resonators 20. It is to be appreciated that, while the embodiments depicted in the figures show two resonators 20, alternative embodiments (not shown in the figures) may include a single resonator of the type described above, wherein the single resonator may be positioned on either the upper inner surface or the lower inner surface of the junction 18. For the purpose of simplicity, the present document will describe the embodiment in which the waveguide circulator 10 includes pair of radial composite resonators 20.

Each resonator 20 may be comprised of a composite made of a centrally disposed ferrite element 30 and a solid dielectric layer 32 (shown in FIGS. 8 and 9) disposed concentrically with and adjacent externally to the centrally disposed ferrite element 30. The junction 18 has an upper inner surface and a lower inner surface positioned in an opposing relationship to one another. The composite resonators 20 are in a spaced-apart, opposing relationship, and are centrally disposed and arranged symmetrically with respect to the three waveguide ports 12, 14 and 16.

In the example shown, the radial composite resonators 20 are affixed atop respective mounting pedestals 22 of the waveguide circulator 10. The radial composite resonators 20 may be fastened to their respective mounting pedestals 22 using any suitable adhesive or glue. In specific practical implementations, silicone-based adhesives may be used to affix the radial composite resonators 20 to the pedestals 22. The mounting pedestals 22 are formed on respective ones of the upper and lower inner surfaces of the junction 18. It will be appreciated that the mounting pedestals 22 may be formed as integral parts of the upper and lower inner surfaces of the junction 18 such that, in practice, the upper and lower inner surfaces of the junction 18 are defined by the mounting pedestals 22. The mounting pedestals 22 hold each of the respective radial composite resonators 20 in place, and form an electrical wall by making contact with the radial composite resonators 20. This arrangement provides a resonator with both a top and bottom electrical wall and a magnetic wall positioned at the midpoint between the two ferrite elements.

Shown in FIG. 4 is a front elevation view of the waveguide circulator 10 with a view into waveguide port 12. Views into waveguide ports 14 and 16 would be similar and thus are not shown here for the purpose of conciseness. In the embodiment depicted, the waveguide ports 12, 14, 16 have a substantially rectangular cross section, defined by a base wall 24, an upper wall 26 and two side walls 28. Each of the waveguide ports 12, 14, 16 has a height Hₚ measured between the base wall 24 and the upper wall 26 and a width Wₚ measured between the two side walls 28. As will be appreciated by the person skilled in the art of microwave devices, the specific dimensions of the waveguide ports are dependent upon the frequency of the signal the circulator is intended to be use for.

Although the waveguide ports shown are of a generally rectangular cross section, it should be appreciated that waveguide ports of other cross sections (such as square or circular) may also be contemplated in alternative implementations.

As mentioned previously, the radial composite resonators 20 are positioned atop the mounting pedestals 22 at the junction 18. Each mounting pedestal 22 has a height Hₘ measured from a respective one of the base wall 24 and the upper wall 26 of the waveguide ports. A ratio Hₘ/Hₚ of the height Hₘ of the mounting pedestal 22 over the height Hₚ of the waveguide ports may take on various values and will be to a certain extent a design choice made by a person skilled in art in order to influence certain operational characteristics of the circulator such as, for example, the frequency of operation. The selection of a specific ratio Hₘ/Hₚ may be performed in accordance with any suitable technique known in the art.

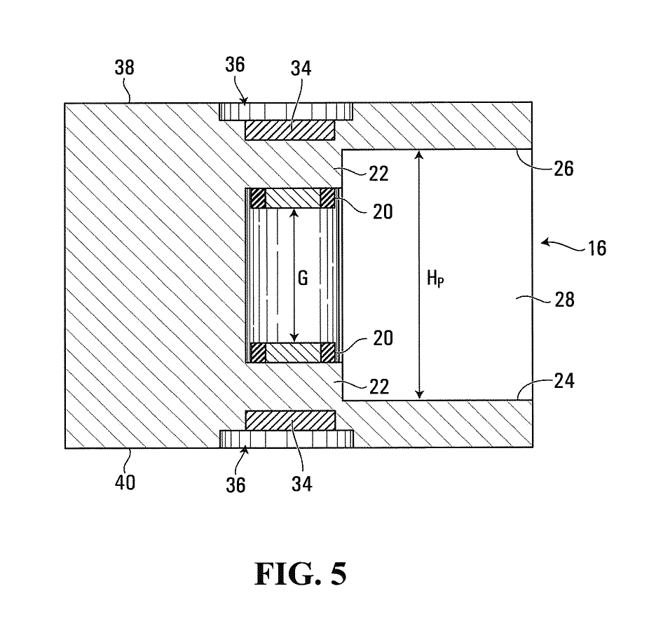

The resonators 20 are positioned in a spaced-apart opposing relationship with one another and are separated by a gap of dimension G, as shown in FIG. 5. The dimension of the gap G between the resonators 20 affects what is referred to as the “filling factor” and can be used to tune the gain bandwidth and the peak power handling of the design, amongst other. Assuming that the distance between the two mounting pedestals 22 is λ₀/2, the dimension of gap G may be found based on the following relationship:

4(L+S)=λ₀,

where L is the thickness of the resonators 20 and where the gap G between the ferrite elements 30 of the radial composite resonators 20 is equal to 2S.

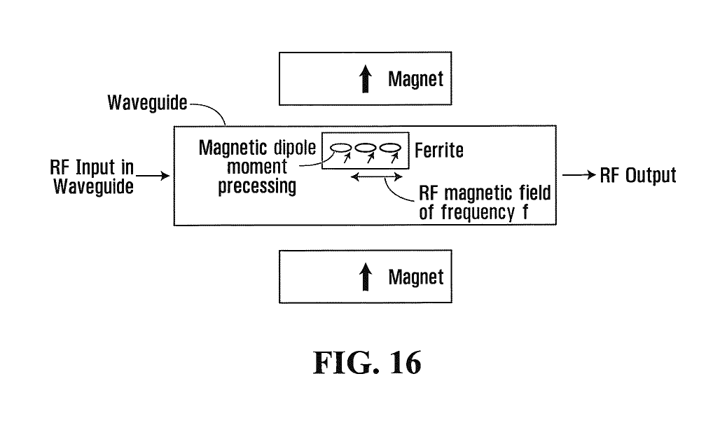

During operation, the radial composite resonators 20 are subjected to the influence of an external magnetic field that is generated by a magnetic field source 34. In this embodiment, the magnetic field source 34 consists of two permanent magnets 34, which are respectively positioned above and below the radial composite resonators 20 in pockets 36 located on respective top and lower exterior surfaces 38, 40 of the waveguide circulator 10. The two permanent magnets 34 may be replaced by electromagnets in some implementations. As illustrated in FIG. 16, when a ferrite element (such as ferrite element 30 in resonator 20) sitting in a metallic waveguide is subjected to an RF field and an external magnetic field, the magnetic dipole moments of the ferrite element will precess. The external magnetic field that is generated by magnets 34 is a uni-directional magnetic field, represented by arrow 42 in FIG. 2, such that wave energy entering each waveguide port 12, 14, 16 will move in a counter-clockwise direction towards its neighboring waveguide port. For example, wave energy from waveguide port 12 propagates to waveguide port 16. Likewise, wave energy from waveguide port 16 propagates to waveguide port 14 and wave energy entering waveguide port 14 propagates to waveguide port 12. In this manner, wave energy is always propagated in a single direction. As such, the waveguide circulator 10 is a non-reciprocal transmitter of electromagnetic wave energy propagating in the waveguide ports. By changing the direction of the magnetic field, it is possible for the wave energy to propagate in the opposite, clockwise, direction. However, regardless of the direction in which the wave energy is propagated, it can only ever travel in one direction at a time.

Radial Composite Resonators 20

FIGS. 8 and 9 are respectively a top plan view and a side view of one of the radial composite resonators 20 of FIG. 2. As shown, the radial composite resonator 20 comprises a composite of a centrally disposed ferrite element 30 and a solid dielectric layer 32 disposed concentrically with and adjacent externally to the ferrite element 30. In particular, as shown, the dielectric layer 32 radially surrounds a peripheral surface of the ferrite element 30. In this embodiment, the ferrite element 30 has a cylindrical disk shape of a diameter D_{f} and comprises a ferrite material having certain ferromagnetic properties. For its part, the dielectric layer 32 is ring shaped to surround the cylindrical disk shaped ferrite element 30 and has an outer diameter D_{d} that is slightly smaller than a diameter of the mounting pedestals 22 and an inner diameter suitable to fit the ferrite element 30. In the embodiment depicted, the ferrite element 30 and the dielectric layer 32 share a common thickness T such that their respective end surfaces are substantially flush with one another. The periphery of the dielectric layer 32, shown in dotted lines in the figures, is beveled in order to reduce the occurrence of arcing. More specifically, and as will be appreciated by the person skilled in the art, during operation, at the edges of the resonator, the electric field may be larger. This can promote the occurrences of arcs or, alternatively, heat portions of the resonators and then create a chip that will detach, which will increase the possibility of arcing. To avoid these, in specific practical implementations, it is suggested to either fillet or chamfer the periphery of the resonators.

In the specific implementation depicted in the figures, the following formula may be referred to when designing the E-plane waveguide circulator in order to determine geometric and material properties of the radial composite resonator 20:

$$\frac{R}{L} = \frac{{( {k}_{0} R )}^{2} {ɛ}_{eff} - {( 1.84 )}^{2}}{{( \frac{π}{2} )}^{2}} ,$$

where R is the radius of the ferrite element (Df/2 in FIGS. 8 and 9), L its thickness (T in FIGS. 8 and 9), k₀=2π/λ₀ where λ₀ is the free space wavelength, and ϵ_{eff} is the effective permittivity of the overall resonator 30. The free space wavelength λ₀ is the length that an electromagnetic field would have should it propagate in air. We use it to normalize the radius, by writing k₀R=2πR/λ₀ instead of simply R. That way we have a value that is normalized, and therefore usable at various frequencies. In the embodiments shown, we have a radial composite resonator 20 having a ferrite element in the shape of an inner disk, and a solid dielectric layer shaped as a dielectric ring, so that the effective permittivity of the radial composite resonator 20 (ϵ_{eff}) may be calculated by combining the permittivity of the two components, for example according to the following formula:

$${ɛ}_{eff} ∼ \frac{π {r}_{in}^{2} {ɛ}_{f} + π ( {r}_{out}^{2} - {r}_{in}^{2} ) {ɛ}_{d}}{π {r}_{out}^{2}} ,$$

where rᵢₙ is the radius of the ferrite element (Df/2 in FIGS. 8 and 9), rₒᵤₜ is the radius of the solid dielectric layer 32 (D_{d}/2 in FIGS. 8 and 9), ϵ_{f} is the permittivity of the ferrite element and ϵ_{d} is the permittivity of the dielectric ring.

This particular configuration of the radial composite resonators 20 may allow for better energy dissipation by the ferrite element 30. More specifically, the dielectric layer 32 may provide an additional contact surface over which the ferrite element 30 can dissipate heat. For instance, the ferrite element 30 may transfer heat directly to the mounting pedestal 22 through its bottom surface and also to the dielectric layer 32 through its peripheral surface. This improved energy dissipation of the ferrite element 30 may allow an increase in the average-power handling of the ferrite element 30. For example, the effective thermal conductivity(σ_{eff}) of the radial composite resonator 20 will be, to first order, a weighted average of the thermal conductivity σ_{f} of the ferrite element 30 and the thermal conductivity σ_{d} of the solid dielectric layer 32, which mathematically can be expressed as:

$${σ}_{eff} ∼ \frac{π {r}_{in}^{2} {σ}_{f} + π ( {r}_{out}^{2} - {r}_{in}^{2} ) {σ}_{d}}{π {r}_{out}^{2}}$$

where rᵢₙ is the radius of the ferrite element (Df/2 in FIGS. 8 and 9) and rₒᵤₜ is the radius of the solid dielectric layer 32 (D_{d}/2 in FIGS. 8 and 9).

In the embodiment presented in the present document, a ratio D_{f}/D_{d} of the diameter D_{f} of the ferrite element 30 over the outer diameter D_{d} of the dielectric layer 32 is approximately about 0.5 and the ratio of the thickness T over the diameter D_{f} of the ferrite element 30 (T/D_{f}) is approximately 0.48. In specific practical implementations, this aspect ratio will typically be selected during the design of the circulator and then, on that basis, the radius, thickness and gap (G) will be derived in order to obtain an overall system that behaves in a desired manner for a desired frequency of operation. For an E-plane circulator of the type contemplated in the present document, an aspect ratio R/L varying between 1.5 to 2.5 has be found to be suitable, however other values may also possible in alternative implementations.

Having a ferrite element 30 with a smaller diameter D_{f} (and hence a larger T/D_{f}) ratio) may present some advantages.

For example, a smaller diameter for the ferrite element 30 may facilitate its external magnetization and may also require a smaller magnet to achieve a desired magnetization level. As a result of the smaller magnet size that may be required, the overall size and/or bulk of the resulting E-plane circulator may be reduced, which may assist in creating a more compact E-plane circulator. The reduced magnet size requirement may be attributable in part to the demagnetization factor N associated with the ferrite element 30, which decreases as the diameter of the ferrite element 30 is made smaller and the thickness thicker.

As will be appreciated by the person skilled in the art, the demagnetization of ferrite element 30 may be different for different directions and may vary in space, because interactions between the magnetic dipoles vary also in space. As such, the demagnetization factor N may accordingly also vary according to direction and space. For the purpose of simplicity and practicality, the axial average demagnetization factor N associated with the ferrite element 30 is most often referred to in practice.

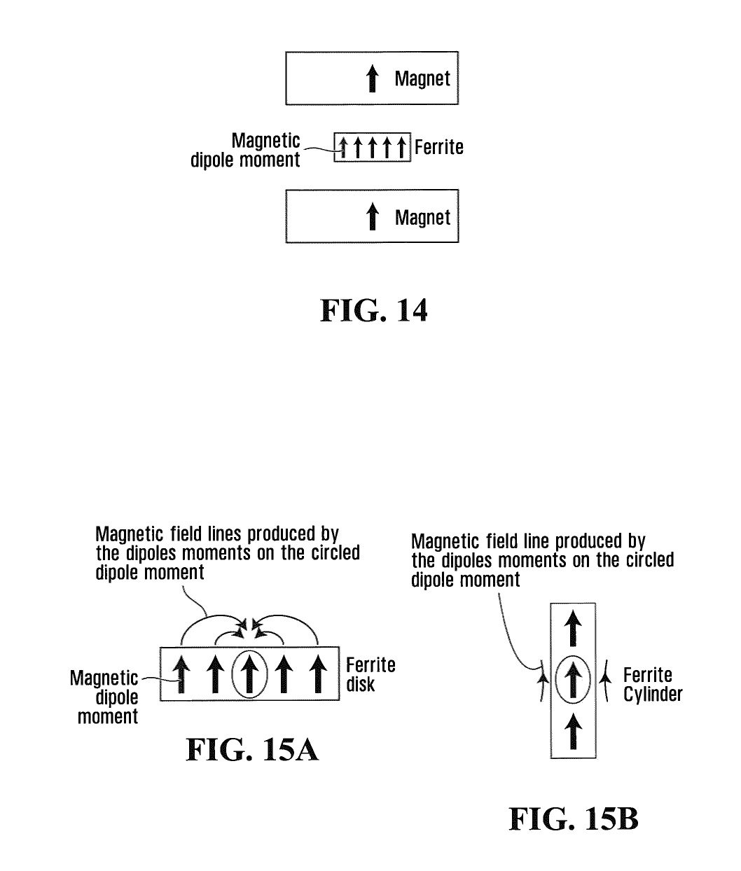

Conceptually, this may be better understood with reference to the diagrams shown in FIGS. 14, 15A and 15B.

As shown in FIG. 14, magnetic dipole moments inside a ferrite element are aligned in a direction corresponding to the direction of an external magnetic field applied to the ferrite element. However, as shown in FIGS. 15A and 15B, these magnetic dipole moments produce a magnetic field on themselves, which can have a direction opposite to that of the applied external magnetic field. The significance of such magnetic dipole moments increases as the diameter of the ferrite element is made larger. This effectively “demagnetizes” the ferrite element. For a strongly demagnetizing scenario, such as the one depicted in FIG. 15A, the demagnetization factor N of the ferrite element approaches “N=1” in the axial direction and approaches “N=0” in the transversal direction. As shown in FIG. 15B, as the diameter of the ferrite element is made smaller, the magnetic field generated by the magnetic dipole moments acts less against the external magnetic field applied to the ferrite element since there are less magnetic dipole moments producing a field opposing the magnetization. For a weakly demagnetizing scenario, such as the one depicted in FIG. 15B, the demagnetization factor N of the ferrite element approaches “N=0” in the axial direction and “N=0.5” in the transversal direction. Thus, a ferrite element having a low demagnetization factor N essentially can reach magnetization saturation using an external magnetic field having a lower intensity than a ferrite element having a high demagnetization factor N. Mathematically, the external magnetic field required to saturate a ferrite element (Hₛₐₜ) is proportional to the demagnetization factor N of the ferrite element and the Mₛ is the level of magnetization saturation and can be expressed as:

Hₛₐₜα Mₛ*N

As can be appreciated from the above, the lower the demagnetization factor N, the lower the required intensity Hₛₐₜ of the external magnetic field that needs to be applied, which may permit smaller magnets to be used.

Another advantage of having a ferrite element 30 with a reduced or smaller diameter D_{f} is that it may allow the use of a more compact magnet while still allowing a relatively uniform magnetization to be achieved over the extent of the ferrite element 30. In the embodiment described, the magnets 34 are disk-shaped permanent magnets having a diameter D_{mg}. In a non-limiting implementation, the ratio of the diameter D_{f} of the ferrite element 30 over the diameter D_{mg} of the magnet 34 (D_{f}/D_{mg}) is selected to be at most 0.7. In other words, the diameter D_{mg} of the magnet 34 is at least about 1.5 times greater than the diameter D_{f} of the ferrite element 30. This difference in size between the magnets 34 and the ferrite elements 30 of the radial composite resonator 20 allows for a more uniform magnetization of the ferrite elements 30.

It will be appreciated that the specific dimensions and shapes of the radial composite resonators 20 used may vary significantly in different embodiments and will depend on multiple design choices that may be made by the person skilled in the art in view of the teachings of the present application.

For instance, while embodiments of the radial composite resonators 20 have been described in which the ferrite element is a ferrite disk and in which the solid dielectric layer is a dielectric ring shaped to surround the ferrite disk, it is to be appreciated that the radial composite resonators 20 in alternative implementations can be of a variety of shapes and/or sizes. For example, in some embodiments, the radial composite resonators can be of a triangular, hexagonal, pentagonal or any suitable arbitrary shape. In a specific implementation in which the ferrite element 30 has a triangular shape, the solid dielectric layer 32 may have a complementary triangular inner surface for surrounding a periphery of the triangular ferrite element 30 and an outer peripheral surface of any suitable arbitrary shape.

Optionally, the radial composite resonator 20 may further include a dielectric stack (not shown in the figures) engaging at least a portion of the top surface of the ferrite element 30. In such a configuration, the ferrite element 30 may transfer heat to the dielectric stack via contact with its top surface. This improved energy dissipation of the ferrite element 30 may affect the effective thermal conductivity (σ_{eff}) and may thus further increase in the average-power handling of the ferrite element 30. The shape, size and composition of the dielectric stack may vary between implementations.

In addition, while the embodiments depicted in FIGS. 2 to 6 show resonator 20 mounted atop the pedestals 22, other alternative configurations are possible. For example, rather than being mounted atop the pedestals 22, the radial composite resonators 20 may be partially embedded within a recess formed on the surfaces of the mounting pedestals 22. In such a configuration, each radial composite resonator 20 may be positioned within such recess such that a first portion of the radial composite resonator 20 projects from the recess and into the junction 18, and a second portion of the radial composite resonator 20 extends into the recess of the mounting pedestal 22. A layer of solid dielectric may be disposed between the second portion of the radial composite resonator 20 (i.e., the portion that extends into the recess) and a surface of the recess formed in the mounting pedestal 22. Such an arrangement may allow increasing the dimension of the gap G between the resonators 20, which may help increase the power handling capability of the circulator. An example of a circulator configuration in which a resonator is embedded within a recess formed on a surface of the circulator junction is described in U.S. patent application Ser. 14/223,628, the contents of which are incorporated herein by reference.

In addition, while in the embodiments depicted in the figures, the waveguide circulator 10 has been shown with mounting pedestals 22 having a height H_{M} (for example see FIG. 4), it is to be appreciated that in some alternative implementations, the mounting pedestals 22 may be omitted (or alternatively may be considered to have a height H_{M} equal to zero). In such implementations (not shown in the figures) the surfaces of the pedestals 20 would lie at the same levels as the lower and upper surfaces of the junction 18, which would in turn lie at the same levels at the base wall 24, an upper wall 26 of waveguide ports 12 14 16.

Operation of the Circulator Above Ferromagnetic Resonance

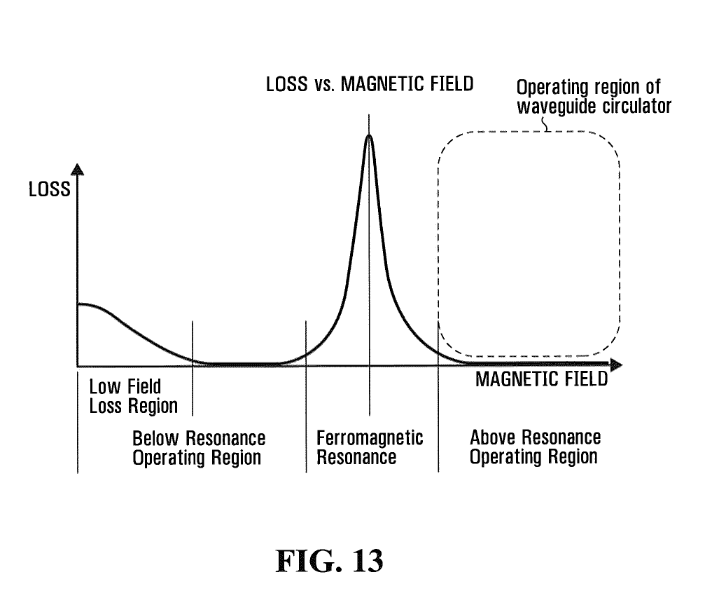

Shown in FIG. 13 is a graph showing energy loss of the ferrite elements 30 as a function of the magnitude of an external magnetic field applied by the magnets 34. As shown, the waveguide circulator 10 described above operates in a region above a ferromagnetic resonance associated with the ferrite elements 30 of the radial composite resonator 20 for a given operating frequency.

A brief explanation of ferromagnetic resonance will be presented simply to facilitate understanding of the concepts presented herein. Ferromagnetic resonance of a ferrite element occurs when the magnetic dipole moments of the ferrite element are caused to resonate in response to a given magnitude of an external magnetic field applied by magnets and a given frequency of RF magnetic field. This resonance of the ferrite element is characterized by a strong absorption of energy by the ferrite element and a maximum amplitude of precession by its magnetic dipole moments. Mathematically, for an axially magnetized ferrite disk, the resonance condition can be expressed as follows,

$${f}_{r} \approx 28 ( {H}_{0} + ( \frac{1 - 3 N}{2} ) {M}_{0} ) ,$$

where fᵣ is the resonance frequency (GHz), H₀ is the magnitude of the external magnetic field at resonance (T), M₀ is the magnetization of the ferrite element (T), and N is the demagnetization factor described above. The above equation (the Kittel equation) can be re-written as following to obtain the magnitude of the external magnetic field at resonance,

$${H}_{0} \approx \frac{{f}_{r}}{28} + ( \frac{3 N - 1}{2} ) {M}_{0}$$

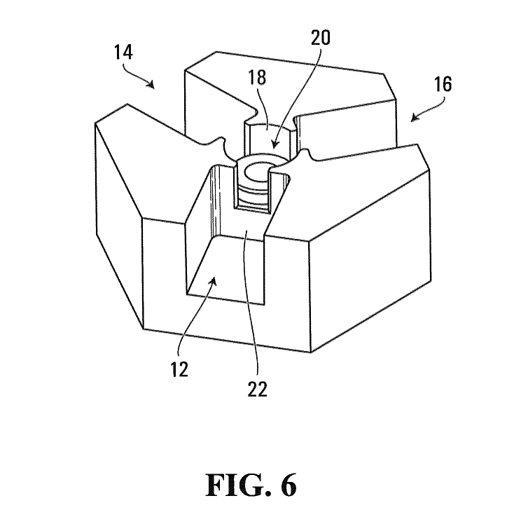

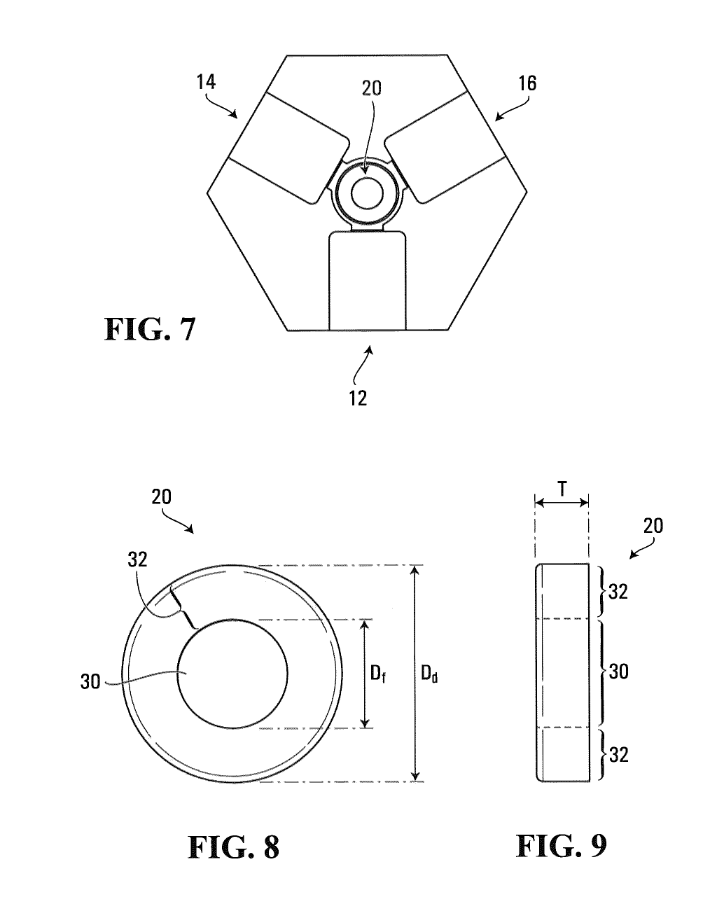

It will be appreciated that a smaller demagnetization factor N, as is obtained by the configuration of the radial composite resonators 20 for example, is associated with a lower resonance magnetic field H₀. If the magnetic field produced by the magnets is smaller than H₀, then the waveguide circulator is said to operate below resonance. Conversely, if the magnetic field produced by the magnets is greater than the resonance magnetic field H₀, then the waveguide circulator is said to operate above resonance. In the case of the waveguide circulator proposed by the present document, an embodiment of which was described with reference to waveguide circulator 10 (components of which were shown in FIGS. 2 to 9), the magnitude of the magnetic field applied by the magnets 34 is greater than the resonance magnetic field H₀ of the radial composite resonators 20 and thus the waveguide circulator 10 operates above resonance.

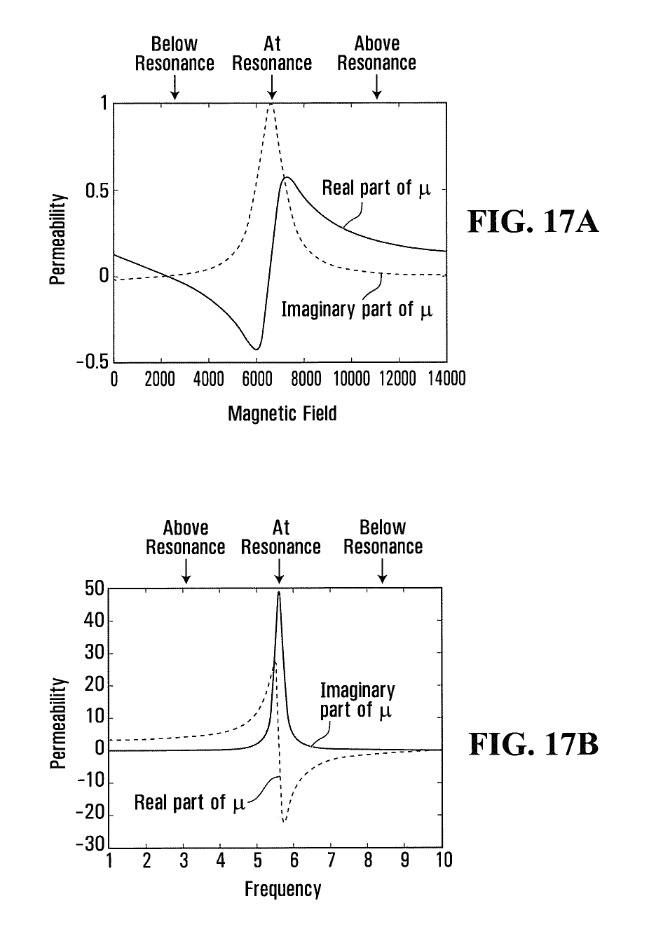

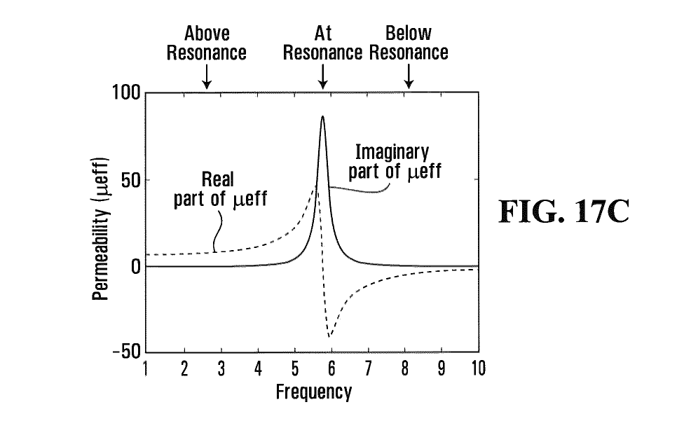

As will be explained in greater detail below, the permeability μ of the ferrite element 30 is affected by the magnitude of the magnetic field applied by the magnets and, as such, the operation of the waveguide circulator 10 in the operating region above ferromagnetic resonance is influenced by the change in permeability μ of the ferrite element. This will best be understood with reference to FIG. 17A, 17B and 17C.

FIG. 17A shows a graph depicting the real and imaginary parts of a permeability μ of a ferrite element as a function of the magnitude of an applied magnetic field. The permeability of the ferrite element defines how strongly the ferrite material reacts to the applied magnetic field. The imaginary part of the permeability is associated with losses (RF energy that is absorbed by the ferrite element and dissipated as heat) whereas the real part of the permeability is associated with the functional aspects of the ferrite element which allows it to interact with the RF magnetic field and create the gyrotropy necessary for the waveguide circulator to operate. As will be appreciated, the waveguide circulator operates above resonance if the magnitude of the magnetic field is greater than the ferromagnetic resonance field of the ferrite element. The waveguide circulator is not operated at resonance since the ferrite element absorbs too much energy at resonance, as can be seen from the graph.

FIG. 17B shows a graph depicting the real and imaginary parts of the permeability μ of the ferrite element as a function of the RF magnetic field frequency. Here, the situation is inverted with respect to FIG. 17A. That is, the waveguide circulator operates below resonance if the resonance frequency is below the frequency of operation, and above resonance if the resonance frequency is above the frequency of operation. This can be explained by the Kittel equation presented earlier.

Of interest for a circulator is the effective permeability of the resonator 20, written μ_{eff}=(μ²−κ²)/μ, where μ is the diagonal component of the permeability tensor and κ is the off-diagonal component of the permeability tensor. The shape is similar to that of the permeability μ of the ferrite element 30.

As will be appreciated by examining FIG. 17C, the real part of the permeability μ_{eff} of the resonator 20 is slightly lower than 1 below resonance, and larger than 1 above resonance. One may consider these features in the design of ferrite element 30 of the resonator 20. For strip-line circulators, the radius R of the ferrite element can generally be considered to be inversely proportional to the effective permeability μ_{eff} of the resonator 20. Mathematically, the relationship between R and μ_{eff} may be expressed as follows (for additional information, the reader is invited to refer to H. Bosma, “On the stripline circulator,” IEEE Trans. Microw. Theory and Techn., vol. 12, pp. 61-72, 1964). The contents of the aforementioned document are incorporated herein by reference):

$$\frac{R}{{λ}_{0}} = \frac{1.84}{2 π \sqrt{{μ}_{eff} {ɛ}_{eff}}} ,$$

where λ₀ is the free space wavelength, and ϵ_{eff} is the effective permittivity of the resonator 20. For circulators operating above resonance, μ_{eff} is larger than 1, so the ferrite element diameter will be reduced compared to below resonance circulators.

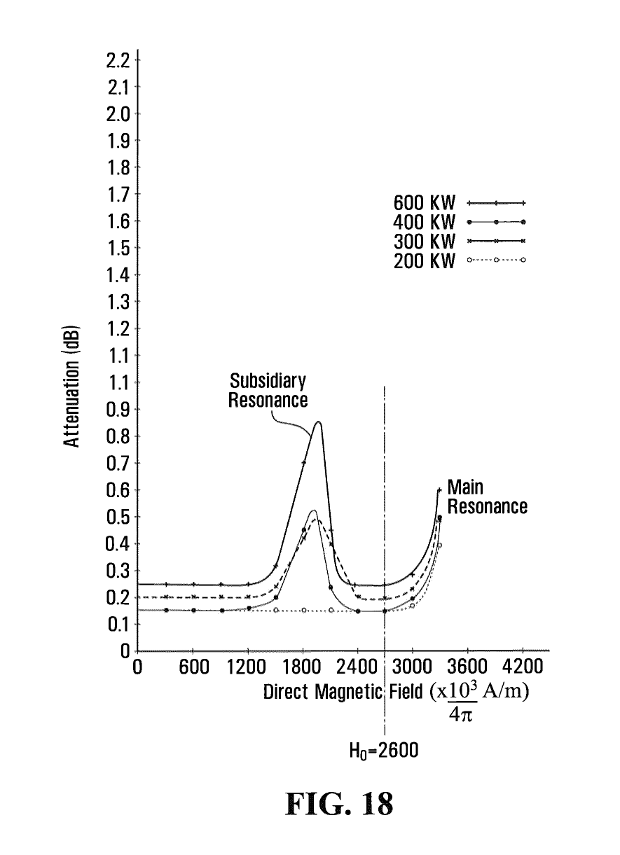

FIG. 18 depicts RF power absorbed (attenuation) as a function of the magnitude of an applied external magnetic field. As can be observed, when the RF power in a waveguide circulator is increased, a subsidiary resonance appears (in addition to the main resonance). For its part, the main resonance peak starts to decrease in amplitude (saturation of the main resonance). The presence of a subsidiary resonance at lower magnetization is due to the excitation of spin waves (i.e., the magnetic moments do not oscillate in unison, rather they oscillate with a phase delay between them).

As the power of the RF wave propagating within the circulator becomes very large, the subsidiary resonance and the main resonance begin to overlap. As such, if it becomes desirable to use circulators at higher peak powers, it becomes necessary to operate between the main resonance and the subsidiary resonance or between the subsidiary resonance and the low field loss region, to avoid absorption of energy by the ferrite elements 30. As power levels increase, such ranges of operation become narrow, if not non-existent. By operating above resonance, the impact on energy absorption by the ferrite due to the overlap between the subsidiary resonance and the main resonance can be avoided. While operating above resonance requires a magnetic field of a greater magnitude (e.g., stronger magnets and/or magnets positioned in closer proximity to one another), it affords a larger practical range of operation.

Simulations of waveguide circulator 10 described above indicate that practical implementations should have power handling capabilities of more than 10 MW at peak-power levels and approximately 8 kW at average power level which would make the waveguide circulator appropriate for some high peak-power applications.

Alternate Configurations

While a specific configuration of an E-place circulator has been described with reference to waveguide circulator 10 (components of which were shown in FIGS. 2 to 9), it is to be appreciated that alternate embodiments of E-plane waveguide circulators of the type contemplated may be configured in many various alternate ways that will become apparent to the person skilled in the art in light of the present description.

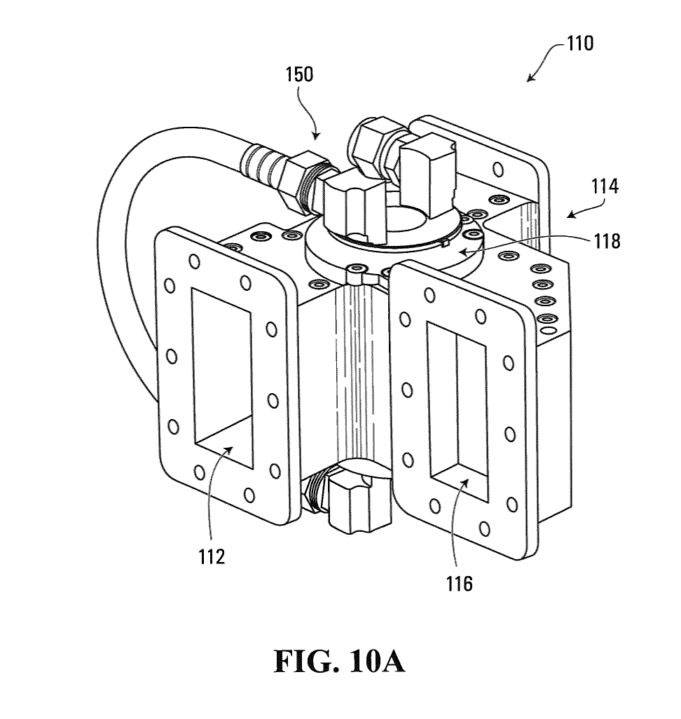

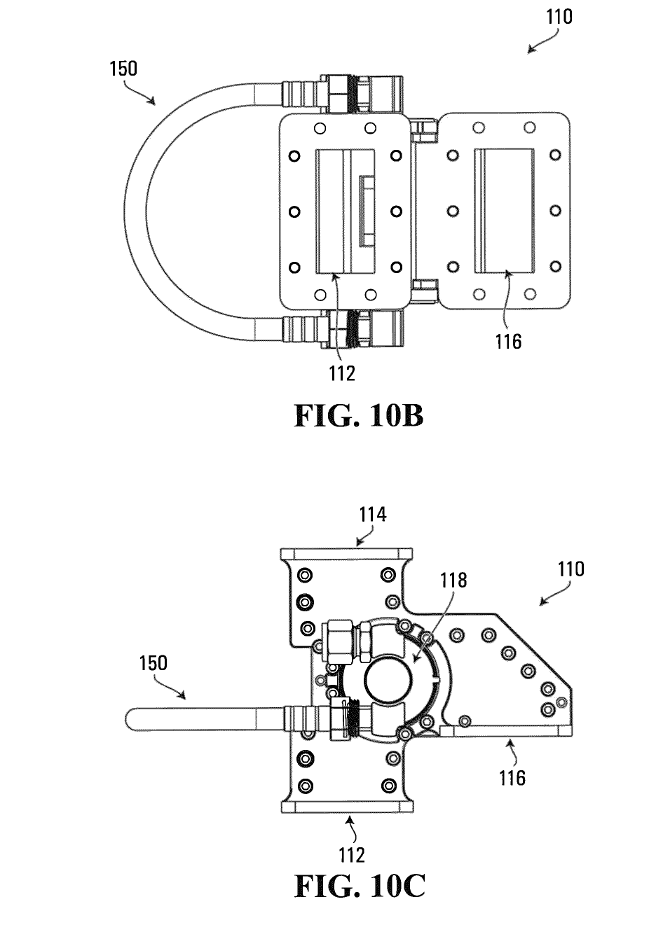

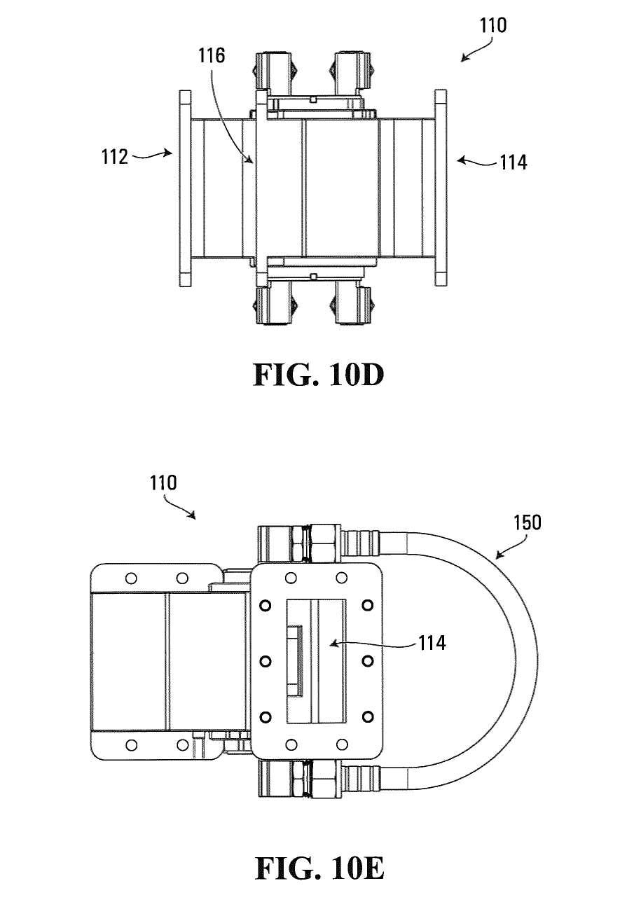

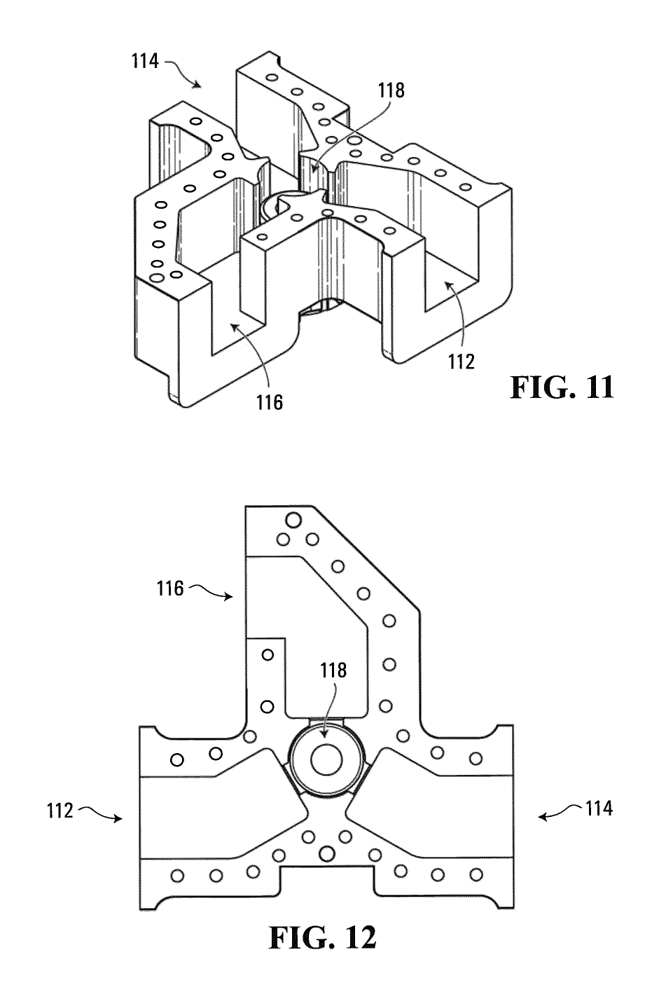

For example, an E-plane waveguide circulator 110 in accordance with another embodiment is shown in FIGS. 10A, 10B, 10C, 10D, 10E, 11 and 12. The waveguide circulator 110 similarly comprises a set of waveguide ports 112, 114 and 116 (analogous to ports 12 14 and 16 shown in FIG. 2) that meet at a junction 118 (analogous to junction 18) where a pair of radial composite resonators (one of which is shown in FIGS. 11 and 12) similar to the resonators 20 described above are positioned. As can be observed, two of the ports 112 116 are oriented such that they are facing substantially the same direction which would allow circuitry connected to these ports to be positioned alongside one another. Such configuration may allow, in some cases, for a more compact layout of the connection circuits and/or termination elements, which in turn would assist in reducing the space required for a circuit incorporating the E-plane waveguide circulator 110. A pair of magnets positioned above and below respective ones of the radial composite resonators is configured to apply a magnetic field of a magnitude above resonance to the radial composite resonators.

In the embodiment shown in FIG. 10A, the waveguide circulator 110 also comprises a cooling module 150 configured to dissipate heat from the junction 118. More specifically, the cooling module 150 comprises circulation piping for circulating a coolant near the junction 118. This may assist in dissipating heat from the resonators at the junction 118 and increase the stability of the circulator 110. Such cooling modules are generally known in the art and thus will not be described further. A similar cooling module may also be used in the waveguide circulator 10 described above.

Manufacturing

In specific practical implementations, waveguide circulators 10 and 110 of the type described in the present document can be manufactured using any suitable manufacturing technique including molding, casting, or machining, among other possible manufacturing techniques. Generally speaking, the waveguide circulators 10 and 110 are made in two separate portions; namely a bottom portion and an upper portion, that are then coupled together in order to form the complete waveguide circulator 10 or 110. The bottom portion and the top portion can be coupled together via welding, bolts, rivets, or any other type of mechanical fastener known in the art. Alternatively, the top and bottom portion may be coupled together by a brazing process.

In accordance with a non-limiting example of implementation, the waveguide circulators 10 and 110 may be made of aluminum. However, it should be appreciated that the waveguide circulators 10 and 110 could be made of any suitable material, such as copper or brass, among other possibilities.

In the above description, only three ports have been shown and discussed in connection with the examples of waveguide circulators 10 and 110 described in the present document. It should however be appreciated that the concepts and features shown and described herein could be equally applied to T-junction circulators, four-port circulators, or circulators having any number of ports.

Waveguide circulators such as the waveguide circulators 10 and 110 described above may be used in a variety of domains. For example, radiotherapy devices used in the medical field to treat cancer or other diseases can use such waveguide circulators in circuit carrying high power RF energy to accelerate electrons or protons which are used to target specific cells in a patient's body (e.g., cancerous cells). In some alternate embodiments, the waveguide circulators 10 110 may be used as part of a satellite communications system. In yet other embodiments, the waveguide circulators 10 and 100 may be used as part of a radar antenna.

The foregoing is considered as illustrative only of the principles of the invention. Since numerous modifications and changes will become readily apparent to those skilled in the art in light of the present description, it is not desired to limit the invention to the exact examples and embodiments shown and described, and accordingly, suitable modifications and equivalents may be resorted to. It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

Although the present invention has been described in considerable detail with reference to certain embodiments thereof, variations and refinements are possible and will become apparent to persons skilled in the art in light of the present description. The invention is defined more particularly by the attached claims.

Listing of claims:

1. An E-plane waveguide circulator for use in a high power microwave circuit, said waveguide circulator comprising:
a) at least three waveguide ports intersecting at a junction, wherein the junction has an upper inner surface and a lower inner surface positioned in an opposing relationship to said upper inner surface; and
b) a radial composite resonator positioned within said junction, said resonator being comprised of a composite made of a centrally disposed ferrite element and a solid dielectric layer disposed concentrically with and adjacent externally to said centrally disposed ferrite element;
c) a magnetic field source for applying an external magnetic field to said radial composite resonator, said external magnetic field having a magnitude above a magnetic resonance associated with the radial composite resonator.

2. An E-plane waveguide circulator for use in a high power microwave circuit, said waveguide circulator comprising:
a) at least three waveguide ports intersecting at a junction, wherein the junction has an upper inner surface and a lower inner surface positioned in an opposing relationship to said upper inner surface; and
b) a radial composite resonator positioned within said junction, said resonator being comprised of a composite made of a centrally disposed ferrite element and a solid dielectric layer disposed concentrically with and adjacent externally to said centrally disposed ferrite element;
wherein in use, an external magnetic field source applies an external magnetic field to said radial composite resonator, the external magnetic field having a magnitude above a magnetic resonance associated with the radial composite resonator.

3. An E-plane waveguide circulator as defined in claim 2, wherein:
a) said ferrite element is a ferrite disk; and
b) said solid dielectric layer is a dielectric ring shaped to surround said ferrite disk.

4. An E-plane waveguide circulator as defined in claim 2, wherein said ferrite element has a triangular shape and wherein said solid dielectric layer has a complementary triangular inner surface for surrounding the periphery of said ferrite element.

5. An E-plane waveguide circulator as defined claim 2, wherein said resonator is positioned on a mounting pedestal formed on one of the upper inner surface and the lower inner surface of the junction.

6. An E-plane waveguide circulator as defined in claim 5, wherein said radial composite resonator is a first radial composite resonator and wherein said mounting pedestal is a first mounting pedestal, said circulator comprising a second radial composite resonator positioned on a second mounting pedestal formed on the other one of the upper inner surface and the lower inner surface of the junction in a spaced-apart opposing relationship with said first radial composite resonator.

7. An E-plane waveguide circulator as defined in claim 5, wherein said resonator is positioned within a recess formed within said mounting pedestal, the resonator including a first portion projecting into the junction and a second portion extending into the recess.

8. An E-plane waveguide circulator as defined in claim 2, said resonator is positioned on one of the upper inner surface and the lower inner surface of the junction.

9. An E-plane waveguide circulator as defined in claim 8, wherein said radial composite resonator is a first radial composite resonator, said circulator comprising a second radial composite resonator positioned on the other one of the upper inner surface and the lower inner surface of the junction in a spaced-apart opposing relationship with said first radial composite resonator.

10. An E-plane waveguide circulator as defined in claim 9, wherein said second radial composite resonator comprised of a composite made of a centrally disposed ferrite element and a solid dielectric layer disposed concentrically with and adjacent externally to said centrally disposed ferrite element.

11. An E-plane waveguide circulator as defined in claim 2, further comprising a cooling module including circulation piping for circulating a coolant near said junction to assist in dissipating heat from said resonator.

12. An E-plane waveguide circulator as defined in claim 2, wherein said magnetic field source includes an electromagnet.

13. A method of using the E-plane waveguide circulator of claim 2, the method comprising applying an external magnetic field having a magnitude greater than a magnetic resonance associated with the radial composite resonator.

14. A radiotherapy device comprising the E-plane waveguide circulator of claim 2.

15. A radar antenna comprising the E-plane waveguide circulator of claim 2.
